(19) United States
(12) Patent Application Publication
(10) Pub. No.: US 2004/0033270 A1
(43) Pub. Date: Feb. 19, 2004

(54) Nano-sized zinc oxide in hygiene products

(76) Inventors: Christian Kropf, Hilden (DE); Claudia Hundeiker, Duesseldorf (DE); Melita Heller, Duesseldorf (DE); Christine Wild, Hilden (DE)

Correspondence Address: Stephen D. Harper, Henkel Corporation, Law Department, 2500 Renaissance Blvd., Suite 200, Gulph Mills, PA, 19406, US

(21) Appl. No.: 10/462,976

(22) Filed: Jun. 17, 2003

Related U.S. Application Data
(63) Continuation of application No. PCT/EP01/14562, filed on Dec. 12, 2001.

(30) Foreign Application Priority Data
Dec. 18, 2000 (DE) DE 100 63 090.1

(51) Int. Cl. A61K 33/32; A61F 13/15; A61F 13/20

(52) U.S. Cl. 424/642; 604/358

(57) ABSTRACT

Hygiene products such as diapers, tampons, pantyliners and the like are produced using zinc oxide in the form of nanoparticles having surfaces that have been chemically and/or physically modified. The surface modification may be carried out using organic compounds such as carboxylic acids, carboxylic acid derivatives, amino acids, hydroxycarboxylic acids, sugar acids, polyglycolic acids, ether carboxylic acids, alkyl halides, or silanes.

This application is a continuation under 35 U.S.C. Sections 365(c) and 120 of International application No. PCT/EP01/14562 (filed Dec. 12, 2001) and claims priority from German application No. 10063090.1 (filed Dec. 18, 2000), each of which is incorporated herein by reference in its entirety.

### BACKGROUND OF THE INVENTION

1. Field of the Invention

The present invention relates to the field of hygiene products, in particular the field of diapers for babies and adults (incontinence products), pantyliners and tampons. In particular, the present invention relates to the use of nano-sized ZnO particles in such hygiene products.

2. Discussion of the Related Art

Hygiene products of the type described above are used to absorb urine, feces, blood and perspiration which the body has excreted. Since the abovementioned excretions create a moist to wet medium, irritations and/or inflammations of the skin, such as diaper dermatitis, may consequently arise. Rubbing of the hygiene product on the skin may additionally speed up the inflammation process.

Baby diapers are already known which contain a skincare lotion on the surface facing toward the skin (nonwoven) (Procter & Gamble). Also known (WO 99/59538) are topical compositions which comprise ZnO with a large surface area (30 to 100 m²/g) and with an average particle size of from 0.1 to 200 μm (in diameter). These compositions are particularly recommended for the absorption of body liquid, e.g. of perspiration, sebum (tallow), urine and water. The effect (e.g. during the treatment of acne or diaper eczema) of the ZnO is attributed to its antibacterial (antiseptic) and also antiinflammatory effectiveness. The latter is described, for example, in Heinrich et al. in Parfümerie und Kosmetik 76, 88-91 (1995).

However, the known products have various quite significant disadvantages: firstly of disadvantageous importance is the fact that the hydrophilic ZnO particles can only be incorporated into hydrophobic compositions with difficulty, if at all (unless the ZnO particles are coated completely with an organic coating, but this in turn hinders its antiseptic and antiinflammatory action). A further disadvantage is that the comparatively large particles or agglomerates on the skin are responsible for an unpleasant feel. A further disadvantage is a large particle requirement and also a poor stability in application systems due to sedimentation of the relatively large particles. Finally, a further disadvantage of the known products is that there is an increased risk of skin irritations as a result of abrasion due to large particles/agglomerates.

Some of these disadvantages can already be avoided by the current prior art. These are all of the abovementioned disadvantages associated with the inadequately small particle size since EP-A 0 791 681 describes ZnO particles with an average particle size of not more than 100 nm which are suitable for coating substrates (such as synthetic, natural and inorganic fibers). The substrates provided with the ZnO particles on the one hand have antibacterial activity and on the other hand have an odor-suppressing activity.

The object which faces the inventors compared with the prior art is to provide hygiene products in the above sense which comprise ZnO as antibacterial and antiinflammatory active ingredient, where this ZnO has a higher effectiveness and is not perceived as unpleasant in tactile terms (i.e. a pleasant wear feel should be achieved at the same time) and is available in stable form in a skincare lipid/wax-based matrix.

In this regard, the inventors of the present invention have tested numerous forms of ZnO for the desired properties and ascertained that various forms of ZnO are able to achieve the set object if they are modified on their surface and are present in a form which is agglomerated as little as possible or not at all.

### SUMMARY OF THE INVENTION

The present invention thus provides for the use of ZnO for the production of hygiene products, where the ZnO is present in the form of nanoparticles which have been chemically or physically modified on its surface. According to preferred embodiments, the hygiene product is a diaper for babies or for adults, a pantyliner or a tampon. According to another preferred embodiment, the chemical or physical modification of the ZnO particle surface takes place with organic compounds, specifically with (a) carboxylic acids (mono-, di- and polycarboxylic acids) or derivatives thereof, such as anhydrides, halides and esters (including the lactones); in particular with stearic acid, palmitic acid, lauric acid, capric acid, caprylic acid, caproic acid, oleic acid, sorbic acid, linoleic acid, linolenic acid, ricinoleic acid, oxalic acid, malonic acid, succinic acid, glutaric acid, adipic acid, citric acid, malic acid, lactic acid, tartaric acid; with (b) amino acids, in particular with the naturally occurring amino acids (Gly, Ala, Val, Leu, Ile, Phe, Tyr, Trp, Pro, Hy-Pro, Ser, Asp, Glu, Asn, Gln, Arg, Lys, Thr, His, Cys, Met); with (c) hydroxycarboxylic acids and sugar acids, such as glucaric acid, gluconic acid, glucuronic acid; with (d) polyglycolic acids of the general formula HOOC—CH₂—O—(CH₂—CH₂—O)ₙ—CH₂—COOH, where n is zero or an integer from 1 to 100, preferably 1, 2, 3, 4, 5, 6, 7, 8, 9, 10, 11 or 12; with (e) ether carboxylic acids of the general formula R—(O—CH₂—CH₂)ₙ—O—CH₂—COOH, where n is zero or an integer from 1 to 100, preferably 1, 2, 3, 4, 5, 6, 7, 8, 9, 10, 11 or 12, and where R is an alkyl, alkenyl or alkynyl radical, but preferably R=C₆-, C₈-, C₁₀-, C₁₂-, C₁₄-, C₁₆-, C₁₈-alkyl, -alkenyl or -alkynyl; with (f) alkyl halides; or with (g) silanes of the type (OR)₄₋ₙSiR′ₙ, where R is an alkyl radical, preferably R=methyl, ethyl, propyl, isopropyl, butyl, t-butyl, and R′ is an organic, in particular an aliphatic, radical having functional groups such as —OH, —COOH, ester, amine or epoxy, where preferably R′=C₆-, C₈-, C₁₀-, C₁₂-, C₁₄-, C₁₆-, C₁₈-alkyl, -alkenyl or -alkynyl, aminopropyl, N-aminoethyl-3-aminopropyl, n- or isopropyl-N,N,N-dimethyloctadecylammonium chloride, nor isopropyl-N,N,N-trimethylammonium chloride, n- or isopropylsuccinic anhydride.

The present invention further provides a method for the production of hygiene products, where the ZnO is applied in the form of nanoparticles which have been chemically or physically modified on its surface to the surface of the hygiene product.

The present invention further, finally, provides a hygiene product containing ZnO, where this is chemically or physically modified on its surface.

### DETAILED DESCRIPTION OF PREFERRED EMBODIMENTS OF THE INVENTION

The average primary particle size of the ZnO nanoparticles (diameter) according to the present invention is in the range 1-100 nm, preferably in the range 1-50 nm or 5-40 nm and 10-20 nm. Particularly preferred values (ranges) for the average particle size are 5 to 20 nm, 10-25 nm or 15-35 nm.

The specific surface area of the particles is at least 10 m²/g, preference being given to values of at least 40 m²/g or at least 100 m²/g.

Accordingly, the present invention relates to hygiene products or parts thereof (which are in contact with the skin, in particular nonwoven materials) which contain nano-sized ZnO particles (ZnO nanoparticles) and which, due to these particles, have an antibacterial (antiseptic) and/or antiinflammatory action. These so-called ZnO nanoparticles (or nanoparticles for short) are preferably those modifications of ZnO which are in the form of nanoparticles which have been chemically or physically modified on the surface. According to a particularly preferred embodiment, the chemical or physical modification of the ZnO particles takes place with a carboxylic acid or one of its derivatives (such as anhydride, halide or ester), with an amino acid, with a hydroxycarboxylic acid or a sugar acid, with a polyglycolic acid of the general formula HOOC—CH₂—O(CH₂—CH₂—O)ₙ—CH₂—COOH, with an ether carboxylic acid of the general formula R—(O—CH₂—CH₂)ₙ—O—CH₂—COOH, with an alkyl halides or with a silane of the type (OR)₄₋ₙSiR′ₙ. Particularly preferred modifying agents are carboxylic acids, in particular fatty acids. Very particular preference is given to stearic acid.

In principle, “ultrasmall” particles (nanoparticles) have properties which differ fundamentally from those of larger particles. Under certain circumstances, they do not scatter light since they are significantly smaller than the wavelength of the light. They can thus produce transparent formulations if they are dispersed to primary particle size. They have a very large specific surface area (10-300 m²/g) and therefore also a high reactivity.

To completely develop their properties according to the invention, the nanoparticles must be smaller than 100 nm. Preferably, particle sizes between 2 and 60 nm are striven for. A further essential criterion for the grade according to the invention of the nanoparticles is a narrow particle size distribution such that the particles are present in as monodisperse a form as possible. In other words, the particle agglomeration should be controlled in order to avoid excessive agglomeration.

In order to be able to utilize the potential of the nanoparticles according to the invention in an optimal manner, production methods are required which allow the preparation of relatively large amounts of nanocrystalline substances with a controlled particle size and narrow particle size distribution. The expenditure on apparatus must be reasonable in order to be able to keep the costs low. Such methods are known in the prior art, but will nevertheless be outlined briefly below in order to better illustrate the present invention.

Firstly, the nano-sized particles must be produced, which must then be further treated in order to control particle agglomeration. For this reason, the intention is to describe below in each case firstly those production methods and then treatment or modification methods which suppress agglomeration. The nanoparticles are used for hygiene products according to the invention thus in a form which has been chemically or physically modified on its surface.

The production methods for nanoparticles (quite generally) based on inorganic materials (oxides, nitrides, metals etc.) can essentially be divided into syntheses via liquid phases (which include the sol/gel process, the precipitation reaction and microemulsion) and gas phase methods.

Liquid Phase

In the sol/gel process, hydrolyzable molecular starting compounds (e.g. ZnCl₂ or Zn(OPr)₂, where OPr is OC₃H₇, i.e. n-propoxy or isopropoxy) are reacted in a controlled manner with water (optionally with the addition of a catalyst) (described by way of overview in EP-B 0 774 443, page 2, [0004] to [0011], albeit for TiO₂, but this has analogous validity for ZnO, and the literature references cited therein). The hydrolysis products then condense to give oxidic nanoparticles. These particles have an extremely large and reactive surface, meaning that OH groups located on the surface of the particles react with one another (condensation) and thus initiate agglomeration. This agglomeration can be prevented by protective colloids or surfactants present during the sol/gel process: the polar groups coat the surface of the particles and thus provide for steric and also electrostatic repulsion of the particles.

A further method of preventing aggregates is the surface modification of the material with carboxylic acids and alkoxysilanes. In this method, the reactivity of the particles is utilized for their (partial) deactivation: the free OH groups are either esterified (carboxylic acids) or silanized. Both cases result in the formation of covalent bonds between the particle surfaces and the surface-active substance. Length and functionality of the organic radical essentially determine the dispersibility of the material in the various media.

In the precipitation reaction, dissolved ions are precipitated by adding a suitable precipitation reagent (often by shifting the pH) (described for TiO₂ in EP-B 0 774 443, pages 3 to 6, [0019] to [0065]). Thermal after-treatment gives crystalline powders, although these normally contain agglomerates. In general, the average particle size, the particle size distribution, the degree of crystallinity, under certain circumstances even the crystal structure and the degree of dispersion can be influenced to a certain extent via the reaction kinetics.

If surface-active substances such as polycarboxylic acids, surfactants or polyalcohols are added during the precipitation process, these coat the surfaces of the growing nuclei and thus prevent uncontrolled further growth of the particles. The surface coating additionally aids the later redispersibility of the isolated powders. This variant of the precipitation reaction is preferred for producing nano-sized powders for this reason and is particularly suitable for the production of ZnO according to the invention.

For microemulsions (ME), the aqueous phases of w/o emulsions are used as reaction spaces for the preparation of nano-sized materials. All of the reactions which serve in aqueous media for the preparation of nano-sized materials can thus in principle also be carried out in microemulsions. This is true particularly of the precipitation reactions and the sol/gel process. The growth of the particles is limited here by the size of the reaction space of the nm-sized droplets. A series of review articles give an overview of ME as reaction media for the preparation of nano-sized materials [e.g. Chhabra et al., Tenside, Surfactants, Deterg. 34, 156-168 (1997); Eastoe et al., Curr. Opin. Colloid Interface Sci. 1, 800-805 (1996); Schwuger et al., Chem. Rev. 95, 849-864 (1995); Lopez-Quintela et al., J. Colloid Interface Sci. 158, 446-451 (1993)].

Further treatment or modification methods, including surface modifiers, which are all suitable for the use according to the invention are described in WO96/34829, WO97/38058, WO98/51747, EP-B 0 636 11 and DE-A 43 36 694.

Gas Phase

In the past 10 years numerous gas-phase processes have been discovered or developed further, meaning that adequate processes are available (e.g. Kruis et al., J. Aerosol. Sci. 29, 511 (1998)). These processes in the gas phase lead, due to the high pressure (with a simultaneously high production rate), to severe agglomeration of the nanoparticles even in the production process, i.e. the reactive particles cluster as a result of sintering operations to give relatively large agglomerates, meaning that it is necessary according to the invention to follow with a method for controlling agglomeration, i.e. a method for modifying the nanoparticles.

In order to be able to assess the grade of the nanoparticles, i.e. inter alia their average particle distribution, various methods are available, the most important of which shall be briefly explained below.

The method of transmission electron microscopy (TEM) requires, as well as a high expenditure on apparatus, also considerable fingertip feeling by the operator and is therefore unsuitable as a standard laboratory method. X-ray diffraction utilizes the evaluation of the width of X-ray diffraction reflections and gives indications as to the size of the primary particles present within the material. The line width arises from the instrumental width (resolution), the broadening based on small particle sizes and the broadening based on microtensions. Assuming that the broadening of the reflections is primarily caused by small spherical particles, the use of the Scherrer equation gives the volume-average size of the investigated crystallites.

To determine the size of colloidal particles, dymamic light scattering is also available, which has in the meantime evolved to become the standard method (Powder and Bulk Engineering, February 1995, 37-45). The advantage of this method is the simple and rapid handling. However, a disadvantage is that the viscosity of the dispersing medium and refractive index of the particle must be known.

The methods of BET isotherm and OH group density can be used as routine methods to further characterize the material.

Recording the BET isotherm gives the specific surface area of the material. In the case of powders with slight degrees of agglomeration, the measured BET surface area should thus deviate only insignificantly from that calculated for isolated particles. Greater differences thus give a direct indication of larger and more dense agglomerates/aggregates (sintering), although the primary particles may, according to X-ray diffraction, be very small.

The determination of the density of hydroxyl groups on the surfaces of the powders gives important information regarding the reactivity and the ability to be functionalized: a low density means that the material was subjected to very high temperatures during synthesis and is at least partially “dead-burnt”. A high hydroxyl group density facilitates functionalization and stabilization of the particles and is therefore preferred.

To determine the OH group density, the powder is reacted with thionyl chloride (exchange OH→Cl) and subsequently quantitatively hydrolyzed (release of the chloride ions). If the specific surface area is known, titration of the chloride ions gives the value for the hydroxyl group density.

The use of ZnO nanoparticles which have been chemically or physically modified on their surface for the hygiene products according to the invention is clearly preferred for various reasons, for example compared with conventional (unmodified) ZnO with an average particle size in the micrometer range (known e.g. from WO99/59538). Firstly, the nano-sized material can be formulated more easily (without resulting in unnecessarily severe sedimentation of the particles), since the modification reduces the hydrophilic property of the ZnO particles and thus facilitates formulation with (hydrophobic) creams. Furthermore, the effectiveness of the ZnO is higher as the result of its enlarged specific surface area for the same amount of nanoparticles used (but this has nothing to do with the modification). Finally, the small particle size also leads to improved sensory properties (tactility) on the skin: no grainy feel is experienced, as is the case with conventional ZnO particles. Moreover, the abrasive property of the particles may be lower for a smaller particle size, and the stress (mechanical damage) to the skin is thus reduced with decreasing particle size.

The properties of ZnO relevant according to the invention are firstly its antibacterial action, and secondly the skin-calming (antiinflammatory) action. Both depend on whether the surface of the ZnO particles as a result of the modification is not a coating in the sense that the nano-sized particles are completely covered, but that Zn ions can be released into the surrounding area by the modified surface. In more concrete terms, modification means the coating of the particle surface with organic compounds which interact via chemical bonds or physical forces with the surface of the particles.

Surface modifiers which can be used according to the invention are, for example, all compounds given as such in the publications WO96/34829 (page 8, line 20, to page 9, line 7), WO97/38058 (page 5, line 28, to page 6, line 17), WO98/51747 (page 5, second paragraph, to page 8, first paragraph), EP-B 0 636 111 (column 3, line 38, to column 4, line 56) and DE-A 43 36 694 (column 6, lines 1/63). Compounds preferred for the modification are, in particular,

(a) carboxylic acids (mono-, di- and polycarboxylic acids) or derivatives thereof, such as anhydrides, halides and esters (including the lactones); in particular stearic acid, palmitic acid, lauric acid, capric acid, caprylic acid, caproic acid, oleic acid, sorbic acid, linoleic acid, linolenic acid, ricinoleic acid, oxalic acid, malonic acid, succinic acid, glutaric acid, adipic acid, citric acid, malic acid, lactic acid, tartaric acid;

(b) amino acids, in particular the naturally occurring amino acids (Gly, Ala, Val, Leu, Ile, Phe, Tyr, Trp, Pro, Hy-Pro, Ser, Asp, Glu, Asn, Gln, Arg, Lys, Thr, His, Cys, Met);

(c) hydroxycarboxylic acids and sugar acids, such as glucaric acid, gluconic acid, glucuronic acid;

(d) polyglycolic acids of the general formula HOOC—CH₂—O—(CH₂—CH₂)ₙCH₂—COOH, where n is preferably 1, 2, 3, 4, 5, 6, 7, 8, 9, 10, 11 or 12;

(e) ether carboxylic acids of the general formula R—(O—CH₂—CH₂)ₙ—O—CH₂—COOH, where preferably R=C₆-, C₈-, C₁₀-, C₁₂-, C₁₄-, C₁₆-, C₁₈-alkyl, -alkenyl or -alkynyl;

(f) alkyl halides;

(g) silanes of the type (OR)₄₋ₙSiR′ₙ, where preferably R=methyl, ethyl, propyl, isopropyl, butyl, t-butyl, and R′ is an organic, in particular an aliphatic, radical with functional groups such as —OH, —COOH, ester, amine or epoxy, where preferably R′=C₆-, C₈-, C₁₀-, C₁₂-, C₁₄-, C₁₆-, C₁₈-alkyl, -alkenyl or -alkynyl, aminopropyl, N-aminoethyl-3-aminopropyl, n- or isopropyl-N,N,N-dimethyloctadecylammonium chloride, n- or isopropyl-N,N,N-trimethylammonium chloride, n- or isopropylsuccinic anhydride.

Other modifiers are surfactants, such as fatty alcohol (FA) derivatives and alkyl polyglucosides (APGs), polymers, such as polyethylene glycols, polypropylene glycols, polyvinyl alcohols, polyvinylpyrrolidone, polyvinyl butyrols or polyaspartic acid, or protective colloids (e.g. gelatin, starch, dextrin, dextran, pectin, casein, gum arabic) and derivatives thereof or mixtures of these.

As is also described in the abovementioned publications, the modification is carried out, depending on the solubility of the substance used for the modification, in water, alcohol (ethanol, n-propanol, isopropanol, propylene glycol), ether (tetrahydrofuran, diethyl ether) or an aprotic solvent (LM), such as hexane, cyclohexane, heptane, isooctane, toluene.

The powder to be modified is dispersed in the LM and where appropriate freed from water residues by boiling on a water separator. The modification reagent is then added and heated under reflux to a temperature between RT and the boiling point of the LM (at atmospheric pressure). Water which forms is optionally separated off using the water separator. The powder is then separated off, for example by means of filtration or centrifugation, from the suspension, washed and optionally dried (drying cabinet, freeze-drying).

The nanoparticles which have been chemically or physically modified on their surface are applied to the hygiene product by methods known from the prior art, for example by impregnation (foulard), roll application or spraying of the hygiene product with a solution/suspension of the finish containing the nanoparticles and subsequent drying.

The nanoparticles can be suspended or dissolved either in anhydrous or in aqueous systems. Both the anhydrous and also the aqueous systems can on the one hand be composed of hydrophobic components, but on the other hand also of hydrophilic components in order to give the hygiene products a hydrophilic or hydrophobic behavior necessary for the various areas of application. If the nonwoven is to absorb liquid, it is provided with a hydrophilic finish; if, by contrast, it is to repel liquid, it must be hydrophobic. Thus, the middle section of a top sheet (uppermost nonwoven of a diaper) is hydrophilic in order to be able to absorb the liquid and to convey it to the lower layers. The outer part of the top sheet, by contrast, is hydrophobic in order to prevent leakage. For both areas, however, an antibacterial and antiinflammatory finish is desired.

The nanoparticle content of such an (abovementioned) finish is in the range from 0.1 to 50% by weight, preferably in the range from 0.5 to 30% by weight, particularly preferably in the range from 1 to 10% by weight.

A further method of applying the nanoparticles to the hygiene product consists in incorporating the nanoparticles into a (skincare hydrophobic) lotion, preferably based on wax, which is applied to the nonwoven material/the fabric sheet. The waxes can be applied during the production of the nonwoven or during the production of the ready-to-use hygiene product (e.g. diaper).

This embodiment is particularly preferred since the content of the nanoparticles in the lotion is less than in the case of the finish (since the application amount of lotion is greater), and is in the range from 0.1 to 10% by weight, preferably in the range from 0.1 to 8% by weight.

### EXAMPLES

### Example 1

### Modification of Nano-Sized (Nano)ZnO with Stearic Acid

60 g of nano-sized ZnO were dispersed in 250 ml of n-octane and freed from adhering water (ca. 1 ml) using a water separator. 10.7 g of stearic acid (98% strength) were then added and the mixture was boiled under reflux for 5 h. During this time, a further 0.5 ml of water was separated off. The resulting nano-sized ZnO powder chemically or physically modified on its surface was then separated off by means of centrifugation, washed with n-octane and dried firstly in air, then for about 8 h at 50° C. in a convection drying oven.

### Example 2

### Modification of Nano-Sized ZnO with Ether Carboxylic Acid

As the ether carboxylic acid R—(O—CH₂—CH₂)_{2.5}—O—CH₂—COOH (R=C₁₂₋₁₄) comprises water as a result of the preparation, 2.7 g of AKYPO RLM 25 (92% strength, trade name from Kao) were firstly dissolved in 200 ml of n-hexane and boiled using a water separator until the water had been completely separated off (the above formula is the description of the average degree of polymerization of the EO groups). 92 g of nano-sized ZnO were then dispersed into this solution and boiled at reflux for 4 h. Water which forms (2.8 ml) was separated off as before. The modified powder was then separated off by filtration, washed with n-hexane and dried for 4-5 h at 50° C. in a convection drying oven.

### Example 3

### Investigations on a Human Three-Dimensional Skin Model

A PIT (phase inversion temperature) cream with conventional ZnO or with nano-sized ZnO which had been coated with stearic acid was prepared. These creams were investigated on a human three-dimensional skin model (Matek Corp., MA Ashland, USA) with regard to their influence on the vitality or on the release of inflammation mediators (interleukin-1α, prostaglandin E2).

Demineralized water (aqua demin.) was applied to four skin models. All of the other skin models were incubated with 80 μl of a 0.16% strength Na lauryl sulfate (SDS) solution for one hour (37° C., 5% CO₂, 90% rel. atmospheric humidity). The skin models were then washed with phosphate buffer and then PIT cream 1 (with conventional ZnO) and PIT cream 2 (with stearic acid-coated nano-sized ZnO) were applied. Four-fold determinations were carried out in each case. As the control, cortisone cream (SDS/aqua demin.) was applied to the four skin models, and aqua demin. (aqua demin./aqua demin.) was applied to four skin models.

After an incubation for 24 hours (37° C., 5% CO₂, 90% rel. atmospheric humidity), the skin models were again washed with phosphate buffer. The skin was then investigated by means of MTT assay (methylthiazoletetrazolium) with regard to its vitality, and in the medium the release of the inflammation mediators interleukin 1-α and prostaglandin E2 was determined.

TABLE 1
PIT zinc oxide cream for experiments on human skin models

| | Cream 1 | Cream 2 |
|---|---|---|
| Ingredients | (% by wt.) | (% by wt.) |
| INCI | 1 | 2 |
| Dicaprylyl ether | 12 | 12 |
| Decyl oleate | 5 | 5 |
| Cetearyl alcohol | 4 | 4 |
| Hydrogenated palm glycerides | 2 | 2 |
| CETEARETH 20 | 2.5 | 2.5 |
| Conventional ZnO (not nanoized, | 3 | — |
| predispersed in water) | | |
| Nanoized ZnO (modified with | — | 3 |
| stearic acid, predispersed in | | |
| dicaprylyl ether) | | |
| Glycerol | 5 | 5 |
| Water | 66 | 66 |
| Phenoxyethanol, methylparaben, | 0.5 | 0.5 |
| ethylparaben, propylparaben, | | |
| butylparaben | | |

The emulsions were prepared in a 2-step process. The ZnO was predispersed either in dicaprylyl ether (2) or in water (1).

The inflammation mediators were determined by means of ELISA assay (Enzyme Linked Immuno Sorbent Assay).

Result:

The treatment of the skin models with Na lauryl sulfate solution and then with aqua demin. (SDS/aqua demin.) led to a reduction in the vitality of the skin models and to increased release of interleukin-1α and prostaglandin E2. In the case of the treatment of the skin models with cortisone cream following incubation with Na lauryl sulfate solution, the release of prostaglandin E2 was significantly reduced, and that of interleukin-1α was only insignificantly reduced. Treatment with PIT cream 1 and with PIT cream 2 led to a slight reduction in the vitality. However, a reduction in the inflammation mediator interleukin-1α was achieved only following treatment with PIT cream 2 which comprised the nano-sized ZnO coated with stearic acid, not with PIT cream 1. Cream 2 also had a tendency to reduce the release of prostaglandin E2 compared to cream 1.

TABLE 2
Vitality of the skin models
Vitality (MTT test)

| | Vitality [% based on | | |
|---|---|---|---|
| | aqua demin./aqua demin.] | | |
| | Individual | | Standard |
| Mixtures | values | Average | deviation |
| Aqua demin/ | 106 | 100 | 6 |
| aqua demin | 95 | | |
| | 96 | | |
| | 103 | | |
| SDS/aqua demin. | 85 | 71 | 11 |
| | 73 | | |
| | 58 | | |
| | 68 | | |
| SDS/cortisone | 105 | 105 | 11 |
| | 111 | | |
| | 89 | | |
| | 113 | | |
| Cream 1 | 94.291 | 90 | 13 |
| | 80.149 | | |
| | 79.007 | | |
| | 105.621 | | |
| Cream 2 | 71.366 | 83 | 10 |
| | 80.676 | | |
| | 84.892 | | |
| | 95.784 | | |

TABLE 3
Release of the inflammation mediator interleukin-1α
Interleukin 1-α

| | interleukin-1α [pg] | | |
|---|---|---|---|
| | Individual | | Standard |
| Mixtures | values | Average | deviation |
| Aqua demin/ | 13.922 | 23 | |
| aqua demin | 10.099 | | |
| | 24.031 | | |
| | 41.977 | | |
| SDS/aqua demin. | 143.957 | 140 | 10 |
| | 126.615 | | |
| | 149.783 | | |
| | 141.052 | | |
| SDS/cortisone | 108.868 | 136 | 18 |
| | 147.839 | | |
| | 142.665 | | |
| | 142.988 | | |
| Cream 1 | 158.563 | 134 | 20 |
| | 133.975 | | |
| | 108.553 | | |
| | 133.654 | | |
| Cream 2 | 66.087 | 69 | 7 |
| | 72.112 | | |
| | 77.271 | | |
| | 62.198 | | |

TABLE 4
Release of the inflammation mediator prostaglandin E2
Prostaglandin E2

| | Prostaglandin E2 [pg] | | |
|---|---|---|---|
| | Individual | | Standard |
| Mixtures | values | Average | deviation |
| Aqua demin/ | 3634.930 | 3999 | 615 |
| aqua demin | 3434.488 | | |
| | 4108.748 | | |
| | 4819.096 | | |
| SDS/aqua demin. | 5721.413 | 8107 | 1690 |
| | 8470.543 | | |
| | 9711.644 | | |
| | 8524.521 | | |
| SDS/cortisone | 712.733 | 869 | 323 |
| | 696.211 | | |
| | 1352.960 | | |
| | 712.733 | | |
| Cream 1 | 23194.855 | 14611 | 6252 |
| | 8363.998 | | |
| | 14395.119 | | |
| | 12488.945 | | |
| Cream 2 | 4942.001 | 9518 | 4662 |
| | 15677.425 | | |
| | 7134.079 | | |
| | 10320.418 | | |

What is claimed is:

1. A hygiene product comprising zinc oxide, wherein the zinc oxide is present in the form of nanoparticles which have surfaces which have been chemically or physically modified or both chemically and physically modified.

2. The hygiene product of claim 1, wherein the hygiene product is selected from the group consisting of diapers, pantyliners and tampons.

3. The hygiene product of claim 1, wherein said zinc oxide has antibacterial (antiseptic) and antiinflammatory properties.

4. The hygiene product of claim 1, wherein said nanoparticles have an average primary particle size (diameter) in the range of from 1 to 100 nm.

5. The hygiene product of claim 1, wherein said nanoparticles have an average primary particle size (diameter) in the range of from 5 to 40 nm.

6. The hygiene product of claim 1, wherein said nanoparticles have a specific surface area of at least 10 m²/g.

7. The hygiene product of claim 1 wherein said nanoparticles have a specific surface area of at least 40 m²/g.

8. The hygiene product of claim 1, wherein the chemical or physical modification of the nanoparticle surfaces is carried out using at least one organic compound selected from the group consisting of carboxylic acids, carboxylic acid derivatives, amino acids, hydroxycarboxylic acids, sugar acids, polyglycolic acids of general formula HOOC—CH₂—O—(CH₂—CH₂—O)ₙ—CH₂—COOH, where n is 1, 2, 3, 4, 5, 6, 7, 8, 9, 10, 11 or 12, ether carboxylic acids of general formula R—(O—CH₂—CH₂)ₙ—O—CH₂—COOH, where n is 1, 2, 3, 4, 5, 6, 7, 8, 9, 10, 11 or 12 and where R=C₆-, C₈-, C₁₀-, C₁₂-, C₁₄-, C₁₆-, C₁₈-alkyl, -alkenyl or alkynyl, alkyl halides, and silanes of general formula (OR)₄₋ₙSiR′ₙ, where R=methyl, ethyl, propyl, isopropyl, butyl, tert-butyl and R′ is an organic radical with one or more functional groups.

9. The hygiene product of claim 8 wherein chemical or physical modification of the nanoparticle surfaces is carried out using stearic acid.

10. The hygiene product of claim 1, wherein the hygiene product additionally comprises a nonwoven material wherein said zinc oxide is present on a surface of said non-woven material.

11. A method for producing a hygiene product comprising applying nanoparticles of zinc oxide having surfaces which have been chemically or physically modified or both chemically and physically modified to a surface of the hygiene product.

12. The method of claim 11, wherein application of the nanoparticles to the surface of the hygiene product is carried out by impregnation, roll application or spraying of the hygiene product with a solution or suspension containing the nanoparticles and subsequently drying said solution or suspension.
